(19) United States
(12) Patent Application Publication
(10) Pub. No.: US 2026/0069145 A1
(43) Pub. Date: Mar. 12, 2026

(54) AUGMENTED REALITY SCANNING INTERFACE FOR GENERATING PERSONALIZED 3D PRINTED CASTS

(71) Applicant: Gero3D Ltd., Tel Aviv (IL)

(72) Inventors: Eli ZERAH, Atlit (IL); Nadav ICHAY, Matan (IL)

(73) Assignee: Gero3D Ltd., Tel Aviv (IL)

(21) Appl. No.: 19/250,806

(22) Filed: Jun. 26, 2025

Related U.S. Application Data
(60) Provisional application No. 63/664,838, filed on Jun. 27, 2024.
(60) Provisional application No. 63/744,929, filed on Jan. 14, 2025.

(51) Int. Cl. A61B 5/00; A61F 5/01; B33Y 50/00; B33Y 80/00

(52) U.S. Cl. CPC A61B 5/0037; A61B 5/0064; A61F 5/01; A61F 2240/004; B33Y 50/00; B33Y 80/00

(57) ABSTRACT

A method of generating a 3D model of a body part includes scanning the body part with a 3D scanner; during the scanning step, generating, with an augmented reality user interface, an augmented reality view of said body part, wherein the augmented reality view differentiates between portions of the body part that have already been scanned and portions that have not yet been scanned; with guidance from the augmented reality view, completing the scan; and generating the 3D model based on data received from the scan. The augmented reality view is displayed on a practitioner-facing screen incorporated into the scanning device. The method may further include imaging the body part with a thermal imager and overlaying thermal scanning data onto the 3D model to thereby generate a distribution map of thermal sensitivity on the 3D model. The design is then generated with holes situated to maximize thermal comfort.

### RELATED APPLICATIONS

This Application claims the benefit of priority to U.S. Provisional Ser. No. 63/664,838 , filed Jun. 27, 2024, entitled “Personalized 3D Printed Casts Designed to Maximize Thermal Comfort,” and U.S. Provisional Ser. No. 63/744,929 , filed Jan. 14, 2025, also entitled “Personalized 3D Printed Casts Designed to Maximize Thermal Comfort,” the contents of both of which are hereby incorporated by reference as if fully set forth herein.

### FIELD OF THE INVENTION

The present disclosure is generally directed to orthopedic devices, and more specifically, but not exclusively, to systems and methods for preparing 3D printed casts.

### BACKGROUND OF THE INVENTION

Various attempts have been made to develop casts and other orthopedic devices (e.g., helmets, splints) with 3D printing. Generally, such devices are manufactured by generating a 3D model of the body part onto which the cast is to be fitted, computer-generating a template for the cast, and printing the cast. The cast is then affixed to the body. For example, the cast may be formed of two halves which are fitted around the limb and attached to each other with latches or straps.

Various techniques have been considered and are currently in use for scanning body parts in order to generate the 3D model of the body part. These techniques include photogrammetry, structured light sensing, laser, optical scanning, and combinations thereof. Many of these techniques are aggregated and compared in [1].

Augmented reality (AR) is a technology that enhances human perceptions of reality by overlaying digital information onto real-world images captured through devices like smartphones or AR glasses. AR involves integrating computer-generated imagery seamlessly with the user's environment. In recent years, augmented reality has been applied to various medical fields, including orthopedics. For example, the doctor or medical practitioner may be equipped with AR equipment during performance of orthopedic surgery. Studies involving the use of such techniques within orthopedics are summarized in [2].
[1] Silva, Rui, et al. “A review on 3D scanners studies for producing customized orthoses.” Sensors 24.5 (2024): 1373.
[2] Doughty, M., et al. “Augmenting Performance: A Systematic Review of Optical See-Through Head-Mounted Displays in Surgery.” J. Imaging 2022, 8, 203.

### SUMMARY OF THE INVENTION

One common challenge for 3D printed casts is ensuring that the scan is of sufficient quality for generating a model of the limb. Taking structured light sensing as an example, various factors may prevent the structured light sensing from accurately generating a model of the limb. These factors could include: obstruction of the contour of the bone (e.g., due to edema, clothing, or excess hair); incorrect placement of body parts during scanning (e.g., a patient folds a finger, and as a result the finger is missing from the scan); or non-compliance by the patient during scanning (e.g., the patient twitches or otherwise moves the limb). Such factors could necessitate discarding the scan of the limb.

The present disclosure introduces an augmented reality scanning interface that enables improved scanning of limbs and design of 3D printed casts. The scanning interface provides a medical professional with live feedback via an augmented reality (AR) user interface (UI). The augmented reality interface may overlay the practitioner's view of the limb that is to be scanned with an image highlighting portions of the limb that have been successfully scanned, or alternatively that still need to be scanned. The user interface may also communicate whether the medical professional should move the scanner closer to or further from the patient. In addition, the augmented reality interface may indicate if there are unexpected errors or missing components of the scan (e.g., if the patient has folded in a finger). Thus, the augmented reality UI provides the medical practitioner with immediate feedback regarding the scanning process, enabling the practitioner to complete the scanning quickly and accurately.

Thus, in a first aspect, a method of generating a 3D model of a body part is disclosed. The method includes: scanning the body part with a 3D scanner; during the scanning step, generating, with an augmented reality user interface, an augmented reality view of said body part, wherein the augmented reality view differentiates between portions of the body part that have already been scanned and portions of the body part that have not yet been scanned; with guidance from the augmented reality view, completing the scan; and generating the 3D model based on data received from the scan.

Likewise, in a second aspect, a system for generating a design for a 3D printed cast, is disclosed. The system includes a scanning device, wherein the scanning device includes a visual camera, a 3D scanner, a screen, and an augmented reality module, wherein the augmented reality module includes augmented reality software configured to display within the screen an augmented reality view of the body part, wherein the augmented reality view differentiates between portions of the body part that have already been scanned and portions of the body part that have not yet been scanned.

The scanner unit or device used for scanning may be any hand-held device or stationary device capable of generating a digital 3D model of the body part. Conveniently, such scanner units are hand-held and simple to operate. The scanner unit may be a tablet computer, for example, and may include a compact CT scanner, a laser-based scanner, a thermal scanner or any of the scanner units mentioned hereinabove.

The augmented reality view may be displayed on a practitioner-facing screen incorporated into a device used to perform the scan. The device preferably includes the 3D scanner, a camera, and a screen, wherein the camera is configured to image the body part at the same time as the scanner is configured to scan the body part, and wherein the screen is configured to display the augmented reality view is a view of the body part captured by the camera overlaid by an augmented reality indication that a given portion of the body part has been properly scanned.

The augmented reality indication may be a shading of the portion of the body part that has been properly scanned, within the view in the display. The display may also provide additional guidance, such as textual instructions, or instructions regarding whether the operator should change positions (move closer or further).

The scanning interface may be integrated with one or more software modules that are trained with machine learning. One such module is a scanning accuracy module. The scanning accuracy module is configured to compare the scan of the body part with data of prior scans of the same body part, and, on a basis of said comparison, evaluate whether additional scanning is required in order to compete a scan of the body part and/or whether portions of the completed scan should be disregarded. The scanning accuracy module has access to data of prior scans of various limbs and body structures, taken from all angles. As a result, the scanning accuracy module is able to determine whether features of a scan of a limb match expected features of that limb and also whether portions of the limb remain unscanned.

In addition, the scanning accuracy module also is used to evaluate the scan following completion. The module considers the scan as received and compares that scan to data of prior scans of the same body part in the database. If irregular features are identified in the completed scan, these features are excluded from the 3D model. The scan considers jewelry, clothing, chains, bracelets, rings, hair, edema of the limb, etc., and removes these irregular features from the final scan file so as to provide the 3D printer with a scan file suited for printing a cast which will match the injured location/limb.

The system may also include a modeling module configured to generate a design for a cast for the body part in accordance with medical requirements, patient comfort, and patient aesthetic preferences, and thereby to optimize the design and construction of the cast. The optimization includes creating a model of the cast which balances between the properties of weight, shape, pattern and rigidity of the cast based on the projected structural load the cast will need to withstand. Preferably, the cast is as light as possible, while having a suitable pattern, meeting rigidity requirements, and supporting the injured limb/area in the appropriate position. The method may further include printing the cast according to the design.

The system described herein may be utilized not only for generating models of a cast, but also for improving diagnosis. To this end, the system described herein may include a diagnostic module. The diagnostic module is configured to derive diagnostic information from the scan of the body part and to integrate said derived diagnostic information with other diagnostic information, and to share the integrated diagnostic information with the modeling module. The diagnostic module receives as input all preexisting diagnostic information (such as x-rays, CT scans, or doctors'observations). The diagnostic module also receives the 3D scan, which may provide additional information about the exact contours of the limb or injury. The diagnostic module is integrated with the modeling module which generates the model of the cast. The modeling module generates a cast model based on both the preexisting diagnosis and based on information learned from the scan.

Optionally, the casts of the present disclosure feature are adapted to a user's comfort by placing ventilation holes according to a heat map of the limb. While it is commonly assumed that 3D printed casts are more comfortable than fiberglass casts (e.g., because they can get wet, and may have patterned holes for wicking away sweat), 3D printed casts are not typically optimized for user comfort. In particular, systems and methods for generating 3D printed casts generally do not account for thermal comfort of the wearer.

The present disclosure thus introduces systems and methods for manufacturing 3D printed casts and other support devices that are particularly designed to maximize thermal comfort of the user. Specifically, during the design process, and as previously discussed, a scanner may be used to accurately measure the dimensions of the patient's limb. The scanning software preferably has an accuracy of +1 mm, ensuring a precise fit. An infrared image (or other thermal image) may be taken of the limb that is to be casted or fitted. The infrared image may then be overlaid onto a 3D model of the limb, to thereby generate a distribution map of thermal sensitivity on the 3D model of the body part, so that a degree of heat sensitivity is assigned to each portion of the limb. A medical professional inputs other information relevant to the layout of the cast, based on goals of the orthopedic treatment. The 3D printed cast is then designed so that holes in the cast correspond to areas of the limb that are hotter, consistent with strength and size requirements of the cast, thereby maximizing thermal comfort in accordance with the distribution map.

The scanner may be any scanner known and available for measuring a three-dimensional shape of an object. Such scanners include structured light 3D scanners, contact scanners, industrial computed tomography scanning, LIDAR, Time of Flight (ToF) 3D scanners, laser scanners, modulated light 3D scanners, photogrammetry, and others. In some cases, the scanner is a structured light 3D scanner.

The 3D printed casts made according to the systems and methods of the present disclosure may be personalized according to preferences of the patient. The patient may select, for example, the color scheme of the cast and text to include on the cast. A 3D modeling process is performed, to tailor the cast to the patient's unique anatomy. The modeling software's pattern and color integration mechanism ensures that the patient's design choices are incorporated into the final cast model.

A latch system may be formed integral to the 3D printed cast. The latch system may include buckles, slots, or other suitable connecting elements, formed out of the same material as the 3D printed cast itself. The latch system may optionally include tamper proof components, or alternatively may be easily releasable, as desired.

Clinical benefits of the devices include: effective immobilization resulting from a precise fit tailored to the patient's unique anatomy; promotion of skin health due to a breathable design; and enhanced patient compliance owing to comfort, hygiene, and personalized design options. In case of supports, braces and the like the benefit is not only in the effective immobilization, but rather in achieving a better fitted support and for protective device that can improve protection of accurately fitted area/limb.

The term “cast” is used herein as an example of an orthopedic or other suitable stabilization device that may be prepared according to the systems and methods disclosed herein. Without limitation, such devices may include: orthesis devices (that can be printed as single object preferably or it can be assembled from more than one piece which are printed), casts, braces, splints, supports, helmets, corrective devices, and protective body armor. The devices are designed for immobilization and stabilization. The conditions treated include: simple and compound fractures, ligament sprains and tears, post-operative immobilization, and other conditions requiring immobilization of the extremities. Additional conditions treated include conditions which require braces and supports which are not for immobilizing.

The term “cast”may also encompass a prosthesis.

The invention further provides methods and methods of doing business, as well as a cast manufactured by said methods. As demonstrated herein, a medical practitioner or the subject in need of a cast, may generate or receive by any means available a digital 3D model of the cast, obtained by scanning the body part to be suited by the cast, wherein said digital 3D model suffices to generate a data set instructive of transforming by printing of said digital 3D model into the desired cast.

In other words, a medical practitioner or a user having a suitable scanner unit, may generate a digital 3D model of the subject's body part requiring support by way of a cast, e.g., a subject's limb, by scanning the body part and generating a patient specific digital 3D model that can be printed onsite or offsite. All that the medical practitioner or the user needs is a suitable scanner and a suitable scanning software, which can be provided at a minimal cost.

The system described herein may further include a data center for generating and storing 3D models suitable for printing 3D casts, the data center comprising a memory unit for storing said 3D models; and an access interface adapted to support selective access to the 3D model to users, wherein the 3D models are stored in a form permitting the user to download said 3D model and transform same by printing the 3D model.

Methods of the invention may be implemented in a clinical arena, bedside, or in a subject's own home. In some cases, a clinic or another medical arena may be used as a center for generating and storing digital 3D models suitable for generating 3D casts by printing. Each of the digital 3D models may be generated for a particular subject by using a scanning unit that is operable to scan upon demand the body part of the subject onto which a cast is to befitted. A medical practitioner or a user present elsewhere, through the use of an access interface can selectively access a memory unit of the data center to retrieve or download said digital 3D model and transform same by printing into the 3D model.

### BRIEF DESCRIPTION OF THE DRAWINGS

FIG. 1 schematically illustrates steps in a method of manufacturing a 3D printed cast, according to embodiments of the present disclosure;

FIG. 2A schematically illustrates components of a system that may be used in order to design a 3D printed cast, according to embodiments of the present disclosure;

FIG. 2B schematically illustrates an augmented reality overlay of a limb during a scanning procedure, according to embodiments of the present disclosure;

FIGS. 3A-3L illustrate examples of a process of designing and applying a 3D printed cast, according to embodiments of the present disclosure; and

FIGS. 4A-4S illustrate accuracy measurements of models of limbs, measured with the systems and methods of the present disclosure.

### DETAILED DESCRIPTION OF THE INVENTION

The present disclosure is generally directed to orthopedic devices, and more specifically, but not exclusively, to systems and methods for preparing 3D printed casts.

The systems and methods described herein may be applied to patients of most ages. Due to the anatomy of young children, certain casts prepared according to the methods described herein may not be suitable for children aged 5 and under. However, there is no upper limitation for the age of the patient. In addition, the casts designed according to the methods disclosed herein are able to accommodate patients of all weight ranges. Because the casts are individually tailored, they can support and immobilize extremities regardless of a patient's body weight.

Patients benefiting from the casts, protective and body armor described herein are, generally, those with orthopedic conditions requiring immobilization and/or are those used for preventive and protective measures. These include: patients with acute fractures or sprains; post-operative patients requiring stabilization during the healing process; and patients with chronic conditions such as osteoporosis or arthritis. Certain patient conditions, however, may not be compatible with the scanning process described herein. These include patients with open wounds and patients with extreme swelling and deformity in the affected region. Likewise, patients with active skin conditions like eczema, psoriasis, or open sores in the area to be casted need to be considered for appropriateness of a 3D printed cast on a case to case basis.

Similarly, patients requiring a prosthesis may benefit from the technology disclosed herein, as the prosthesis may be tailored to fit on the stump in a way maximizing their comfort and stability.

Notwithstanding any particular use, the casts may be integrally formed with a variety of functional or aesthetic accessories, or may be configured to receive or attached to such accessories.

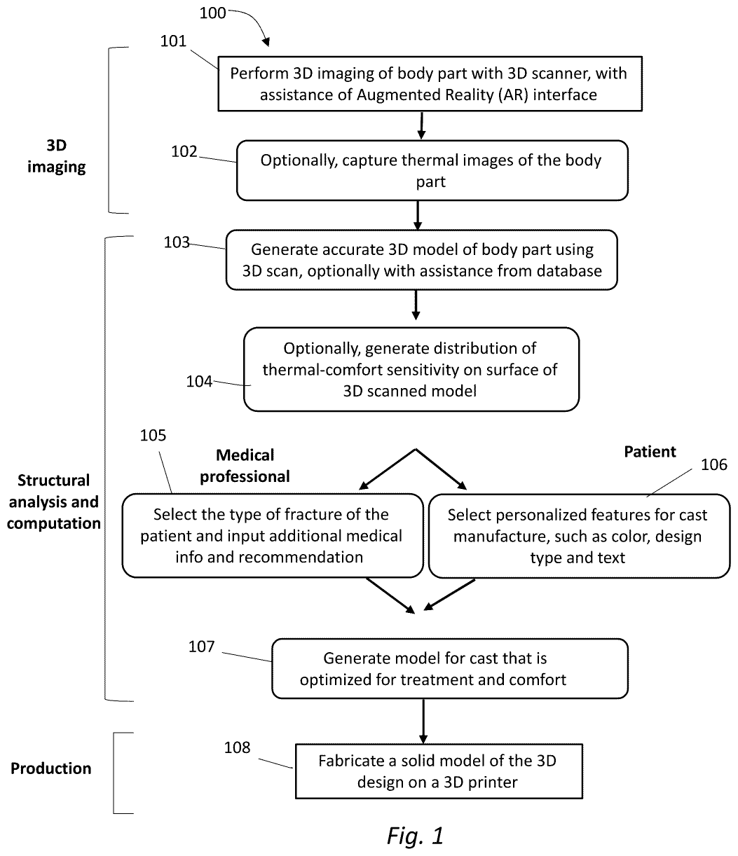

Referring now to FIG. 1, at step 101, a medical practitioner scans a body part with a 3D scanner. For example, the 3D scanner may be a Structured Light Scanner (SLS). A SLS includes a light source and a camera. The light source from the scanner head projects a series of parallel patterns onto the scan target. When the light projects onto the object's surface, the patterns become distorted. The camera captures these images including the distortions, and the images are analyzed to thereby provide the 3D structure of the object. The foregoing is merely one example of a 3D scanner, and any other suitable sensor may alternatively be utilized.

The device used by the medical practitioner for the scanning is equipped with an augmented reality user interface (UI). The augmented reality user interface provides an augmented reality view of the body part being imaged. The augmented reality view differentiates between portions of the body part that have already been scanned and portions of the body part that have not yet been scanned. For example, the device may be a tablet computer which includes the structured light sensor on a patient facing side and a screen on the practitioner-facing side. The screen may thus depict an augmented reality view of the limb that is being scanned, and differentiate between portions of the body part that have already been scanned and portions that have not yet been scanned. In addition, the user interface may provide other instructions to the user, such as whether to stay at the same distance from the patient, to come closer, or to retreat further back. The user interface may also provide confirmation that a scan has been successfully completed.

The scanning process is performed under suitable environmental conditions for generating an accurate scan. The scan is properly performed in consistent and diffused lighting. Direct sunlight or overly bright artificial lights can interfere with the scanning process, leading to inaccurate scans. Ideally, indoor lighting with minimal shadows or glare is recommended. In addition, a neutral and non-reflective background is desirable. This ensures that the structured light patterns are easily distinguishable and not confused with background noise.

Patient cooperation and preparation also is necessary in order to ensure a proper scan. During the scanning process, the patient's extremity should remain stable. Movement can cause disruptions in the structured light patterns, leading to inaccuracies. It is recommended to have the patient seated or supported to minimize movement. In addition, shiny or excessively hairy surfaces can cause reflections or obscure the structured light patterns. In such cases, prepping the area, possibly by shaving or using a matte spray, can be beneficial.

The scanning device is preferably held at a consistent distance and angle relative to the patient's limb, throughout the scanning process. This ensures the most accurate representation of the extremity's dimensions.

At step 102, optionally, thermal images of the body part are obtained with a thermal imager. The thermal imager records the presence of long and medium wavelength infrared radiation that is emitted from different portions of the body part. The thermal imager may be incorporated into the same hardware device as the 3D scanner or may be incorporated into a different hardware device.

There is no particular requirement as to which of steps 101 or 102 is performed first. Theoretically, they may be performed simultaneously.

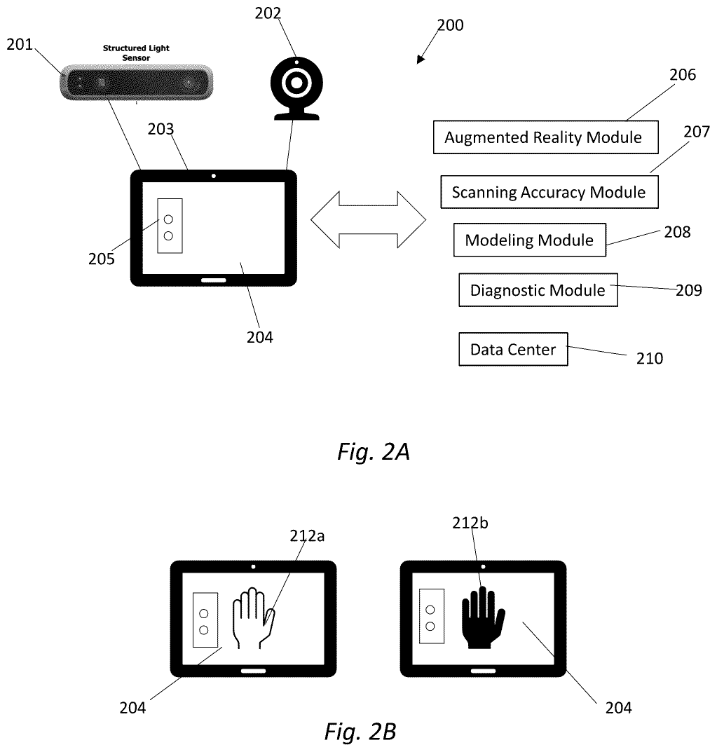

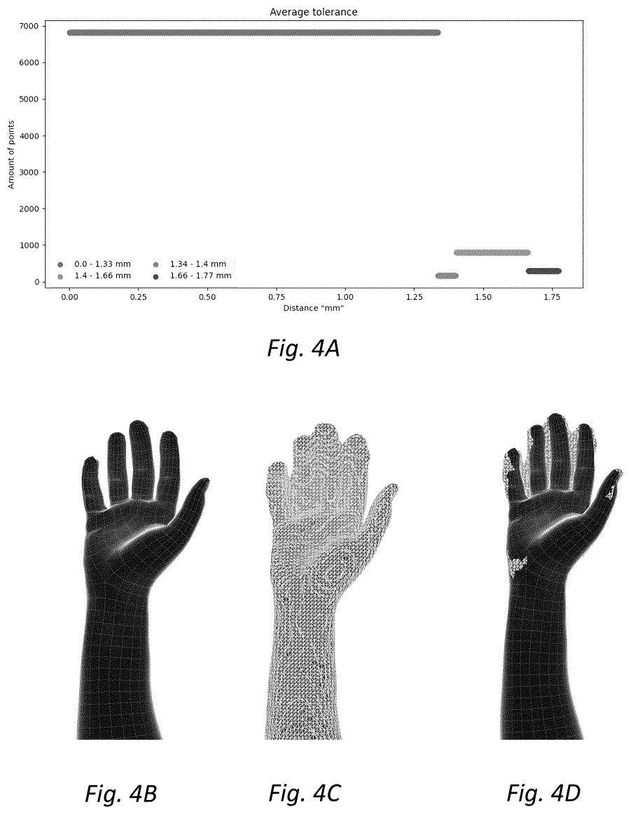

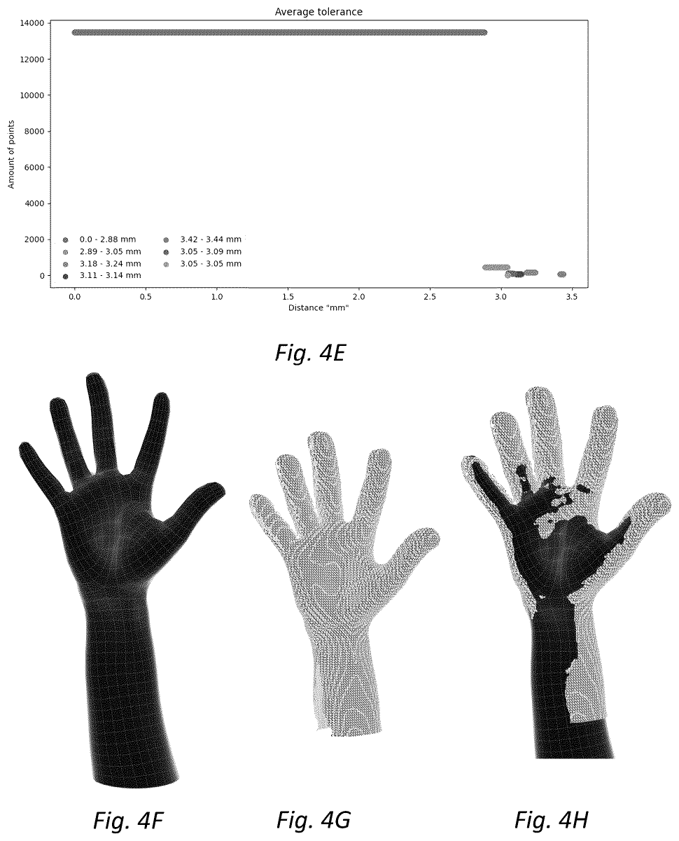

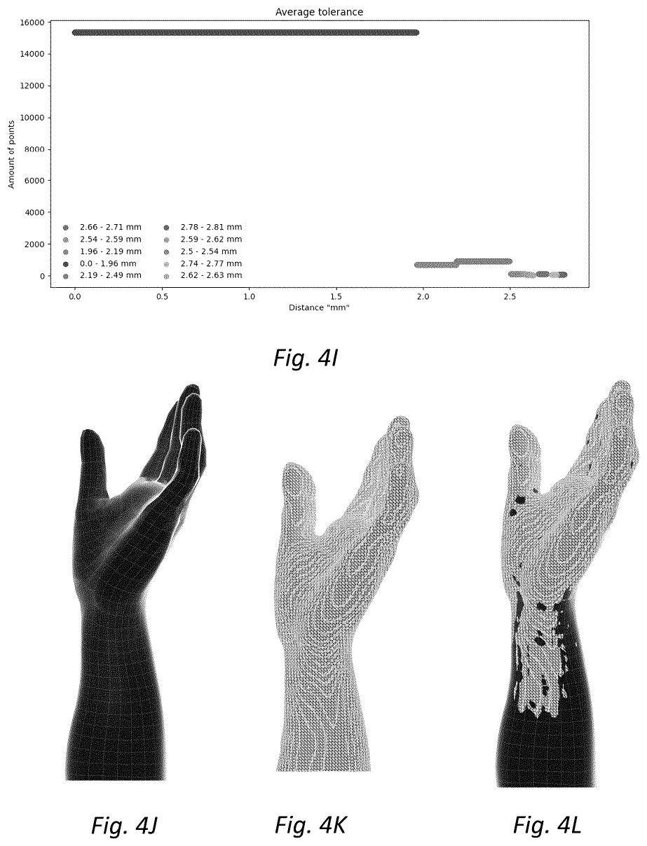

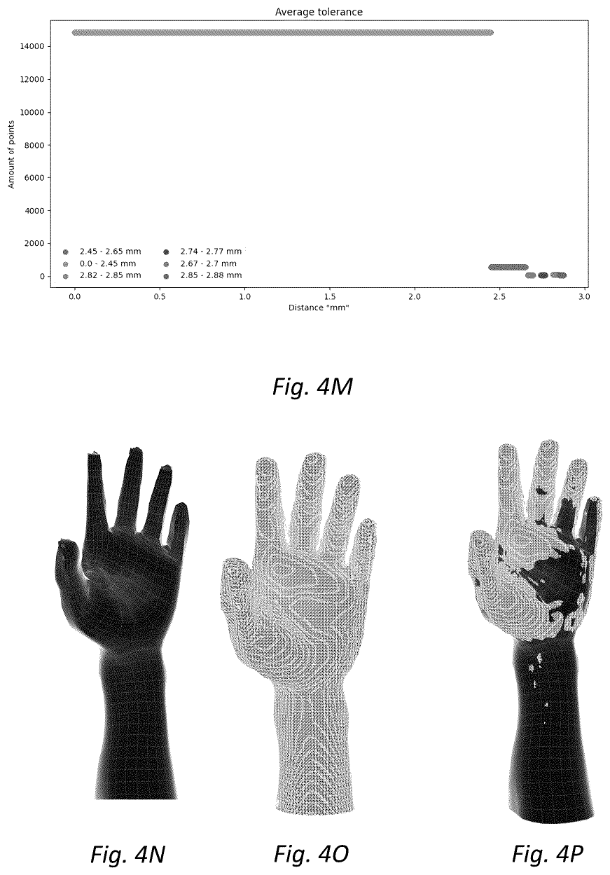

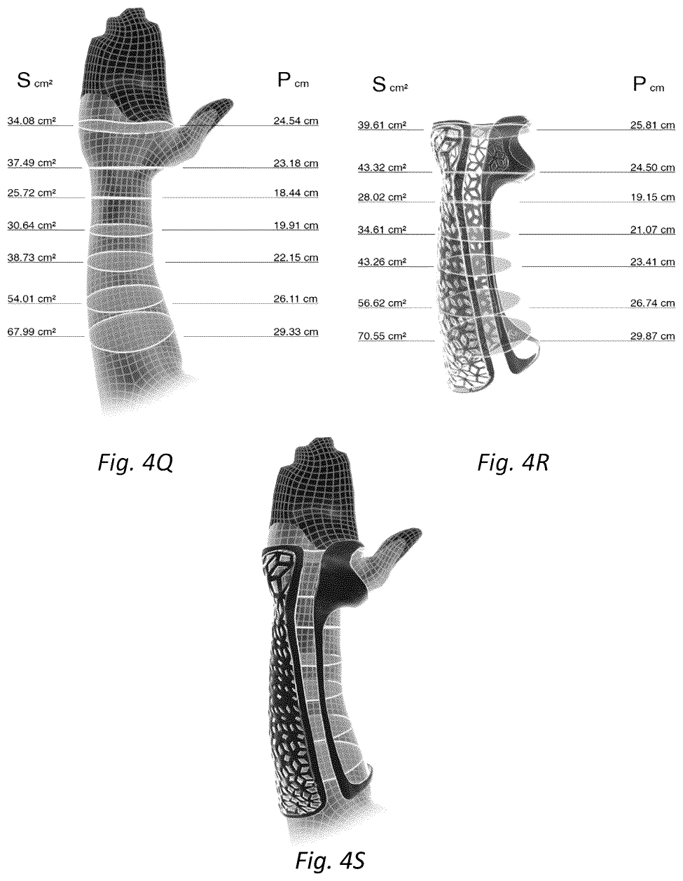

At step 103, data from the structured light sensor is utilized in order to generate a digital 3D model of the limb. This modeling may be performed using a proprietary software module, described herein as a “modeling module.” Experimental results incorporating this proprietary software will be discussed further below, in connection with FIGS. 4A-4S. A scan verification and other post-processing actions may be performed with a “scanning accuracy module” prior to generating the digital 3D model, as will be discussed further herein in connection with FIG. 2A.

At step 104, when a heat scan is performed (per optional step 102), the scanning and thermal imaging data are overlaid so as to generate a distribution of thermal-comfort sensitivity on the surface of 3D scanned model. In this step, a 3D map of the body part is produced, in which each point on the map has a particular value for the relative degree of heat that emanates from that point.

At steps 105 and 106, additional data is input into a computer program for purposes of designing the 3D printed cast. Specifically, at step 105, a medical professional may select a general design for the cast based on the type of fracture that is being treated. The medical professional may also input additional medical information and the recommended treatment. At step 106, the patient (or any other individual) may select personalization features for the cast, such as color, design type, and text to include on the cast.

In exemplary embodiments, on average, steps 101-106 cumulatively take approximately 10 minutes.

At step 107, the 3D model of the limb, thermal comfort sensitivity data (when present), the medical requirements, and the design requests are fed into a processor. Additional inputs from the patient's medical history and scans may optionally be included. For example, results of CT, ultrasound, and X-Ray scans, and other medical data may be considered. In other examples, there are cases in which the injured limb cannot be properly scanned, e.g. due to immobilization of the limb, swelling, edema. Thus, in addition or in the alternative to scanning of the injured limb, a 3D scan may be taken of a non-injured limb. For example, if a fractured arm cannot be properly scanned, the operator of the process may determine that the scan will be taken of the non-injured arm. The system is instructed to convert the 3D scan to a model of the injured limb (e.g. mirror imaging).

In some embodiments, the scan information may also be used to derive diagnostic information. For example, the scan may provide information about the precise contours of the limb, or of the injury thereto.

The processor (and, in particular, a modeling module thereof) analyzes all the inputs, applies a rules-based algorithm, and determines the optimal design for the cast. The rules-based algorithm may operate, for example, by ranking certain priorities (medical effectiveness, comfort, etc.), determining various design options consistent with those priorities, and designing the cast while ensuring that all the priorities are met.

Optimization of the design includes creating a model of a cast which balances between the properties of weight, shape, pattern and rigidity of the cast based on the projected structural load the cast will need to withstand. The modeling module may also factor into the model the heat map scan, when present. Preferably, the cast is as light as possible, has a suitable pattern (optionally, matching the heat map) and rigidity requirements and is shaped to match the cast's functional purpose, i.e. supporting the injured limb/area in the appropriate positioning.

The design of the cast typically includes holes. The holes are designed in accordance with considerations of strength, comfort, and design. The holes may be of any suitable shape, including regular geometric patterns (such as pentagons, hexagonal, circular, etc), Voronoi patterns, and irregular patterns. The holes may be configured at locations selected for maximizing patient comfort in view of heat and in view of patient skin conditions, as discussed.

The casts are typically designed and constructed of a single body which may include hinges and fasteners which are an integral part of the cast. In addition, the cast may include parts which enable the cast to expand and contract in accordance with the required size (an “accordion” element). The fasteners may be for attaching to other supporting devices such as a sling or for the attachment of treatment modules such as TENS (Transcutaneous Electrical Nerve Stimulation) leads.

At step 108, a solid cast or other support device is printed, based off of the design, with a 3D printer. The cast may be made with any suitable 3D printer, such as a powder bed or inkjet printer. The material of the cast may differ depending on the specific type of support device and support role that is desired. For example, a polymeric material such as a nylon may be used for casts and splints, whereas ceramic and metallic materials may be used for braces and protective body armor. Composites may also be used alone or in combination with other materials.

The cast is then affixed to the body of the patient. The cast may be removed and re-applied when needed, e.g. treatment of a wound, showering, examination etc. Optionally, the fasteners are applied in a tamper-proof manner.

FIG. 2A illustrates components of a system 200 that may be utilized to implement the method of the present disclosure. The system includes structured light sensor 201 (which includes a camera) or another type of 3D scanner with a separate camera, and, optionally, infrared heat detector 202. The system further includes a device 203 (e.g. a tablet computer) with a screen 204 including user interface 205. The screen may provide the user an image of what is being captured by the camera and 3D scanner during the scan, overlaid with an augmented reality indication that differentiates between portions of the body part that have already been scanned and portions of the body part that have not yet been scanned. For example, the augmented reality indication may be a shading of the portion of the body part that has been properly scanned, within the view in the display. The user interface 205 may also include dots for signifying instructions to the user (e.g., a dot of one color signifies an instruction to draw closer, and a dot of a second color signifies an instruction to retreat further away from the patient). The user interface may instruct a user to scan at different angles or positions in order to obtain a more accurate scan. The user interface may communicate instructions any other way, e.g. with audio or text-based instruction.

Thus, in preferred embodiments, the system 200 comprises a device that includes the 3D scanner 201, a camera, and a screen 204, wherein the camera is configured to image the body part at the same time as the scanner 201 is configured to scan the body part, and wherein the screen 204 is configured to display the augmented reality view is a view of the body part captured by the camera overlaid by an augmented reality indication that a given portion of the body part has been properly scanned.

The system described herein also includes various software programs, or “modules” which are stored on a non-transitory memory of a computer and are executable to perform various functions, as described herein. The software programs may be encoded on a memory of the device 203 or may be encoded on a memory of a separate device. Typically, the scanning data is uploaded to a cloud-based device for processing. An advantage of running the program on a cloud-based device is that the cloud-based computer may have greater computing capabilities as well as more memory for storage of patient data. The software modules generally constitute computer programs that are trained based on extensive databases using machine learning techniques.

In particular, the systems of the present disclosure may include the following modules:

Augmented Reality Module 206. The augmented reality module correlates the inputs of the camera and the 3D scanner and, on a basis thereof, provides the visual indication and instructions to the user with respect to performance of the scanning, as discussed.

Scanning Accuracy Module 207. The scanning accuracy module is configured to compare the scan of the body part with data of prior scans of the same body part, and, on a basis of said comparison, evaluating whether additional scanning is required in order to compete a scan of the body part and/or whether portions of the completed scan should be disregarded. The scanning accuracy module is trained on a machine learning program which accesses data of prior scans of limbs and various body structures. For example, during the scanning process, the scanning accuracy module may identify “missing” parts (e.g., a folded finger may initially appear in the scan as a missing finger) and instruct the user to further scan in order to obtain a full scan. Following completion of the scanning, the scanning accuracy module may compare the completed scan of the body part with data of prior scans of the same body part. If irregular features are identified in the completed scan, these features may be excluded from the 3D model of the limb. The irregular features may be, for example, clothing, jewelry, excess hair, or edema in the area of the limb. This module may also “fill in” missing parts, e.g. a missing finger, in the event that this was not discovered during the scan.

Modeling Module 208. The modeling module combines all of the inputs (diagnostic inputs, 3D scanning, heat map, and user preferences) to optimize the design of the cast.

Diagnostic Module 209. The diagnostic module integrates all known diagnostic information, including information learned from the scan itself. The sources of diagnostic information may include a medical professional's diagnosis, and a computer-generated diagnosis based on imaging, machine learning, and the like. The diagnostic module shares the diagnosis and data thereof via an API with the modeling module and the modeling module generates a cast model based on the diagnosis and the scan.

Data Center 210. The data center is used for generating and storing 3D computer models (generated by the modeling module) which are suitable for printing 3D casts. The data center includes a memory unit for storing said 3D models; and an access interface adapted to support selective access to the 3D model to users, wherein the 3D models are stored in a form permitting the user to download said 3D model and transform same by printing into the 3D model.

FIG. 2B illustrates schematically an example of the overlaying performed by the augmented reality module. In the left side of the figure, an unscanned limb 212a is shown in display 204 in its typical view as it would appear in a camera. In the right side of the figure, the scanned limb 212b is shown within the display 204 as completely shaded. The shading is added during the scanning process.

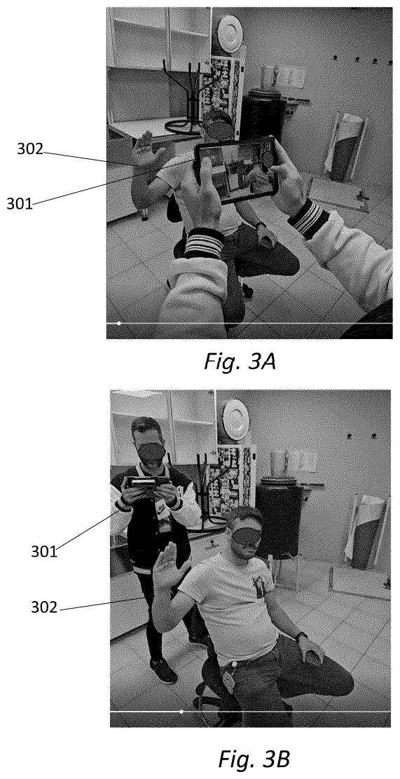

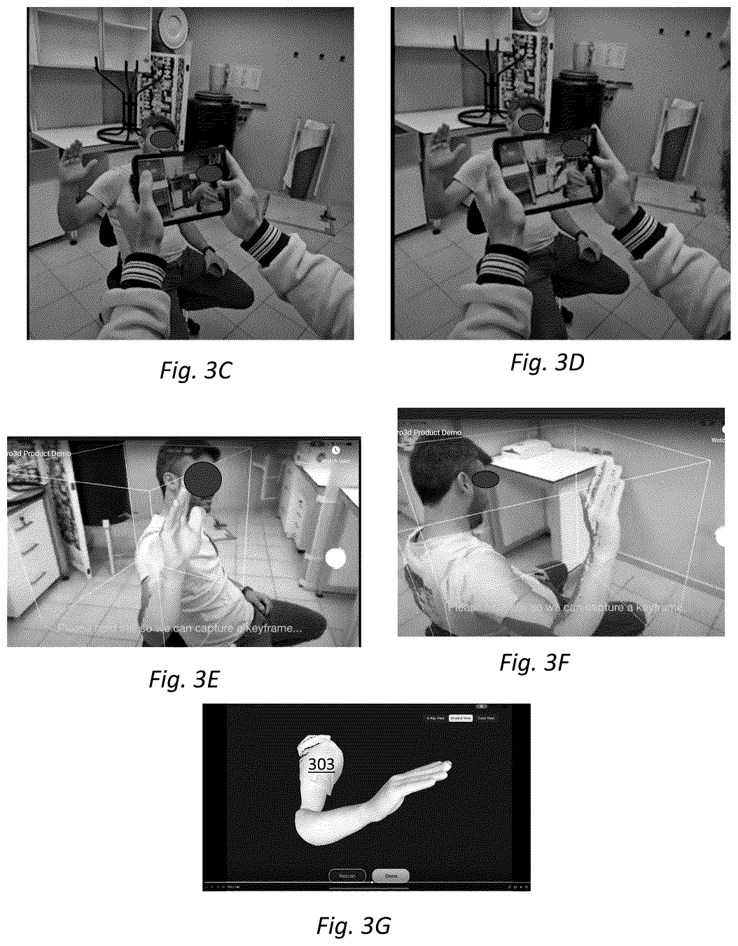

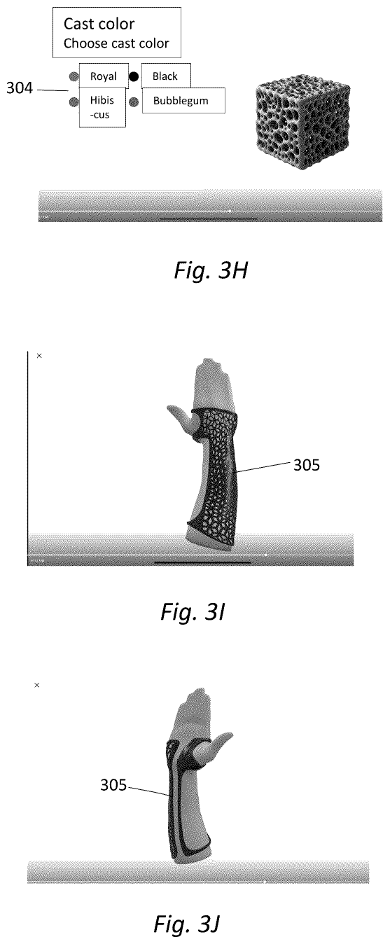

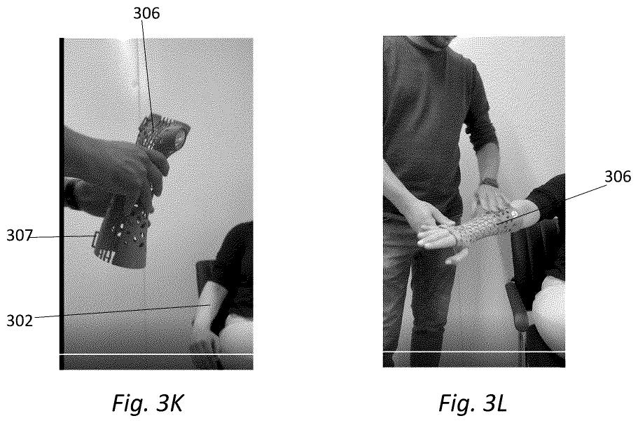

FIGS. 3A-3J illustrate method 100 being implemented on a patient. In FIG. 3A, optionally, a medical professional uses device 301 to capture a heat map of the injured limb 302. The limb 302 is shown with an augmented reality heat map overlaid onto the image of the limb. In the illustrated embodiment, the device 301 is an Apple ipad Mini® 6 tablet computer, with a Structure Pro Sensor for structured light scanning connected to the tablet with a USB-C interface. In FIG. 3B, the medical professional uses the same device 301 to perform 3D mapping of the limb, walking around the limb in order to capture it from all angles. As discussed above, a different device may also be utilized for this function.

FIGS. 3C-3F illustrate the process of the augmented reality module shading the limb as the scanning is performed. In FIG. 3C, during the beginning of scanning, no shading is overlaid onto the limb, although the heat map may be overlaid on the limb, having been retained from the prior step, as shown here. In FIG. 3D, as soon as scanning begins, the portion of the limb that was scanned is shaded in the user display. As the medical practitioner walks around the limb, scanning additional portions thereof, the shading in the limb gradually fills in. This progression is shown in FIGS. 3E and 3F. Also, as shown in FIGS. 3E and 3F, the display may include visual cues to the medical practitioner, including a bounding box around the limb that is being imaged and textual instructions regarding how to perform the scan.

FIG. 3G illustrates a 3D computer model 303 of the limb that may be generated from the 3D mapping. FIG. 3H illustrates an exemplary user interface for selection of color and other aesthetic features of the cast. FIGS. 31 and 3J illustrate different views of a computer model 305 of the cast. FIGS. 3K and 3L illustrate application of the 3D printed cast 306 onto the limb 302 and securing of the cast in place. The cast 306 includes latches 307 formed integral to the cast 306 and which may be closed in a tamper-proof manner.

### Accuracy of Scanning and 3D Modeling

A 3D modeling software was tested for accuracy by capturing images of printed limbs, and by comparing the known dimensions of those limbs to the dimensions that were calculated following scanning and modeling of the limbs using a proprietary scanning application. Statistical methods were used to interpret the accuracy of the scanning and algorithmic processes.

The process involved using the application to scan various phantom limbs. In particular, the challenge was to capture intricate details and dimensions. Each model was scanned multiple times in order to test the consistency of the scanning technology. The scanned dimensions were compared against actual model dimensions. Mean errors and standard deviation were calculated for each data set.

FIGS. 4A-4D illustrate results with regard to scanning a first model of a hand. FIG. 4A represents the summary of the data, FIG. 4B is an image of the model hand, FIG. 4C is an example of a scan of the model hand, and FIG. 4D is an overlay of the scan on the model. The same set of Figures is illustrated for a second model (FIGS. 4E-4H), a third model (FIGS. 4I-4L), and a third model (FIGS. 4M-4P).

In the example of FIGS. 4A-4D, the mean discrepancy was 1.05 mm. The median discrepancy was 0.87 mm, and the standard deviation was 0.76 mm. In the example of FIGS. 4E-4H, the mean discrepancy was 1.79 mm, the median discrepancy was 1.52 mm, and the standard deviation was 1.32 mm. In the example of FIGS. 41-4L, the mean discrepancy was 1.15 mm, the median was 0.91 mm, and the standard deviation was 0.99 mm. For FIGS. 4M-4P, the mean discrepancy was 1.31 mm, the average was 1.17 mm, and the standard deviation was 0.94 mm. Overall, the results indicate a general trend of mean discrepancies ranging from 1.05 mm to 1.79 mm across the different cases. The median values, which are less sensitive to outliers, show a slightly lower range of discrepancies, suggesting a central tendency towards a closer fit. The standard deviations, which represent the variability of the discrepancies, vary across the cases but generally indicate a moderate level of variation in the scanning accuracy. In general, the study demonstrates that the proprietary app possesses a commendable level of accuracy in scanning extremities for the purpose of creating custom 3D-printed casts.

FIGS. 4Q-4S illustrate evaluations of the precision of the algorithm for fitting casts to the scanned dimensions of the human hand. The evaluation was performed by measuring and comparing the area and perimeter across various levels of both the scanned hand and the output cast.

Detailed measurements of area and perimeter were conducted at seven distinct levels on the scanned hand and the corresponding output cast. Each level represents a strategic cross-sectional segment of the hand. The levels were selected to ensure a thorough and comprehensive assessment. The study calculated area and perimeter for each level. These are illustrated in FIG. 4Q for the model of the hand (area on left, circumference on right) and in FIG. 4R for the model of the cast (area on left, circumference on right). FIG. 4S illustrates the model of the cast overlaid onto the model of the hand.

The differences between the model of the hand and cast were measured, and statistical analysis was performed to determine the median, mean, and standard deviation to quantify the overall accuracy. The obtained results showcased remarkable precision in both the area and perimeter measurements: The area and perimeter at each level were closely matched between the scanned hand and the output cast. For area, the median difference was −3.903 cm², mean was −3.965 cm², and standard deviation was −1.351 cm². For perimeter, the median difference was −0.984 cm, mean was −1.156 cm, and standard deviation was −0.316 cm.

These results are highly favorable. The slight differences observed are within an exceptionally acceptable range, indicating high precision in the algorithm's output. The consistency in discrepancies suggests a systematic and reliable performance of the algorithm in all segments of the hand. Furthermore, both the mean and median values point to a minimal gap between the scanned dimensions and the output cast, underscoring the algorithm's effectiveness. The small discrepancies are well within the range of clinical acceptability and unlikely to impact the comfort or effectiveness of the casts.

Features, integers characteristics, described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed. A skilled person will understand additional features and use of the system and method.

1. A method of generating a 3D model of a body part, comprising:
scanning the body part with a 3D scanner;
during the scanning step, generating, with an augmented reality user interface, an augmented reality view of said body part, wherein the augmented reality view differentiates between portions of the body part that have already been scanned and portions of the body part that have not yet been scanned;
with guidance from the augmented reality view, completing the scan; and generating the 3D model based on data received from the scan.

2. The method of claim 1, wherein the augmented reality view is displayed on a practitioner-facing screen incorporated into a device used to perform the scan.

3. The method of claim 2, wherein the device used to perform the scan is a tablet computer.

4. The method of claim 2, wherein a device used to perform the scan includes the 3D scanner, a camera, and a screen, wherein the camera is configured to image the body part at the same time as the scanner is configured to scan the body part, and wherein the screen is configured to display the augmented reality view is a view of the body part captured by the camera overlaid by an augmented reality indication that a given portion of the body part has been properly scanned.

5. The method of claim 2, wherein the augmented reality indication is a shading of the portion of the body part that has been properly scanned, within the view in the display.

6. The method of claim 2, wherein the guidance comprises a textual instruction.

7. The method of claim 1, further comprising, during the scanning step, comparing the scan of the body part with data of prior scans of the same body part, and, on a basis of said comparison, evaluating whether additional scanning is required.

8. The method of claim 1, further comprising, following completion of the scanning step, comparing a completed scan of the body part with data of prior scans of the same body part, and if irregular features are identified in the completed scan, excluding said features from the 3D model.

9. The method of claim 8, wherein the irregular features include one or more of clothing, jewelry, hair, and edema.

10. The method of claim 1, further comprising generating a design for a cast for the body part in accordance with medical requirements, and printing the cast according to the design.

11. The method according to claim 10, further comprising imaging the body part with a thermal imager and overlaying the thermal scanning data onto the 3D model to thereby generate a distribution map of thermal sensitivity on the 3D model of the body part, and generating the design with a plurality of holes situated so as to maximize thermal comfort in accordance with said distribution map.

12. The method of claim 10, wherein the step of generating a design comprises selecting color and text of the cast based on inputted patient preferences.

13. The method of claim 1, further comprising imaging a non-injured body part, and extrapolating data from the imaged non-injured body part to thereby improve the 3D model of the body part onto which the cast is to be fitted.

14. The method of claim 10, further comprising deriving diagnostic information from the 3D model of the body part, and wherein the step of generating a design comprises integrating the derived diagnostic information with additional diagnostic information so that the cast is configured to achieve therapeutic goals.

15. A 3D printed cast made according to the method of claim 1.

16. A system for generating a design for a 3D printed cast, comprising:
a scanning device, wherein the scanning device includes a visual camera, a 3D scanner, a screen, and an augmented reality module, wherein the augmented reality module includes augmented reality software configured to display within the screen an augmented reality view of the body part, wherein the augmented reality view differentiates between portions of the body part that have already been scanned and portions of the body part that have not yet been scanned.

17. The system of claim 16, further comprising a scanning accuracy module configured to compare the scan of the body part with data of prior scans of the same body part, and, on a basis of said comparison, evaluating whether additional scanning is required in order to compete a scan of the body part and/or whether portions of the completed scan should be disregarded.

18. The system of claim 16, further comprising a modeling module configured to generate a design for a cast for the body part in accordance with medical requirements, patient comfort, and patient aesthetic preferences.

19. The system of claim 18, further comprising a diagnostic module configured to derive diagnostic information from the scan of the body part and to integrate said derived diagnostic information with other diagnostic information, and to share the integrated diagnostic information with the modeling module.

20. The system of claim 16, further comprising a data center for generating and storing 3D models suitable for printing 3D casts, the data center comprising a memory unit for storing said 3D models; and an access interface adapted to support selective access to the 3D model to users, wherein the 3D models are stored in a form permitting the user to download said 3D model and transform same by printing the 3D model.
